(19) United States
(12) Patent Application Publication
(10) Pub. No.: US 2022/0165378 A1
(43) Pub. Date: May 26, 2022

(54) SYSTEM FOR AUTOMATIC GENERATING CUSTOMIZED MEDICAL INFORMATION

(71) Applicant: X Intelligence Inc., Zhubei City (TW)

(72) Inventor: Ten-Yi HUANG, Zhubei City (TW)

(21) Appl. No.: 17/526,656

(22) Filed: Nov. 15, 2021

(30) Foreign Application Priority Data
Nov. 25, 2020 (TW) 109215530

(51) Int. Cl. G16H 15/00; G16H 30/40; G16H 10/60; G16H 40/67

(52) U.S. Cl. CPC G16H 15/00; G16H 40/67; G16H 10/60; G16H 30/40

(57) ABSTRACT

The present creation provides a system for automatic generating customized medical information, which includes a storage medium, a processing unit and a user interface. The storage medium stores a patient education database, which further stores a plurality of patient education information corresponding to a plurality of diseases. The processing unit is coupled to the patient education database. According to a diagnostic result information, the processing unit selects the first patient education information corresponding to a first disease from the patient education database, and generates a customized medical information including the first patient education information. The user interface is coupled to the processing unit to receive and display customized medical information. This system saves medical work time, optimizes the medical experience of patient, and reduces medical disputes.

### BACKGROUND OF THE INVENTION

### 1. Field of the Invention

The present invention relates to a medical system, especially to a medical system for generating customized medical information of patients automatically.

### 2. Description of the Prior Art

At present, the information provided by medical institutions to patients, such as detailed diseases information, nursing methods, precautions, etc., are all standardized documents that have been established. These documents may be outdated, inconsistent with the patient's condition, unclear message description, etc. As a result, patients cannot understand their own condition deeply, and they have no idea about the correct caring way, which leads to deterioration of the disease. Otherwise, the information may be too formulaic that leads to the same solution in different condition. Therefore, the patient does not realize the severity, and the medical order cannot be implemented; or, the required medical treatment period gets longer, and medical resources are wasted.

The generation of medical reports needs to be filled out in manually by experienced physicians, but it is a considerable repetitive work for the physicians. The repetitive work takes a large workload and low processing efficiency, and it is prone to mistake, therefore to increase the labor costs. On the other hand, doctors inadvertently use professional medical vocabulary to communicate usually, but people without medical knowledge may not communicate with doctor. They don't even know how to ask questions, and the doctors may not have the patience to explain, either. Medical disputes caused by poor communication between doctors and patients are obviously unavoidable.

Therefore, there is an urgent need in the art for a system that can automatically generate medical information or reports to save time for manual operations.

### SUMMARY OF THE INVENTION

In view of this, the present invention provides a system for automatic generating customized medical information, comprising a storage medium, a processing unit, and a user interface. The storage medium has a health education database. Wherein, the health education database stores a plurality of health education information corresponding to a plurality of medical conditions, the processing unit is coupled to the health education database. Wherein, according to a first medical condition described in diagnostic result information. The processing unit selects a first health education information corresponding to the first medical condition from the health education database, and generates a customized medical information comprising the first medical condition and the first health education information. The user interface is coupled to the processing unit to receive and display the customized medical information.

The system for automatic generating customized medical information further comprises an image analysis device. The image analysis device is coupled to the processing unit. Wherein, the image analysis device is configured to analyze a medical image corresponding to a patient to diagnosis the first medical condition, and to output the diagnostic result information comprising the first medical condition and the medical image. The processing unit is configured to generate the customized medical information comprising the medical image, the first medical condition and the first health education information.

The storage medium of the system further comprises a medical record database. The medical record database is coupled to the processing unit, wherein the medical record database stores basic information of a patient and at least one medical history information. Wherein, the processing unit is configured to revise the customized medical information according to the basic information and the medical history information. Each of the health education information comprises a prognosis information, a symptom information, a medical care information, and a medical order information. Each of the health education information is divided into a plurality of differential health education information according to a clinical difference factor. The processing unit is configured to compare the clinical difference factors with the basic information of the patient and the medical history information, and to select a first differential health education information correspondingly from the first health education information to generate the customized medical information.

The storage medium of the system further comprises a medical personnel database. The medical personnel database is coupled to the processing unit and storing a medical personnel information of a medical personnel. Wherein, the processing unit is configured to select the medical personnel information of the medical personnel corresponding to a patient form the medical personnel database. The customized medical information generated by the processing unit comprises the medical personnel information.

The user interface of the system further comprises a medical affairs interface. The medical affairs interface is coupled to the processing unit for inputting a customized medical order; wherein, the processing unit is configured to revise the customized medical information according to the customized medical order.

The user interface of the system further comprises a patient interface. The patient interface is coupled to the processing unit to receive and display the customized medical information, wherein the patient interface stops displaying the customized medical information after a preset period, and the patient interface is a web browser interface or a mobile application interface.

The storage medium of the system further comprises a report generation model, wherein the processing unit is configured to generate the customized medical information into a customized medical report according to the report generation model, and the user interface is configured to present the customized medical report.

The storage medium stores a command, and the processing unit is configured to execute the command to select the first health education information according to the diagnostic result information and generate the customized medical information.

In summary, the present system greatly reduce the time for medical staff to design, fill in medical information and medical reports, and can be customized to be used in different cases. From the patient's point of view, seeing complete, customized, and easy-to-understand health education information will help to understand their own disease, clearly choose treatment options and take care of body. In particular, patients also see demonstrations, predictions, and attending physicians or related medical personnel from the user interface. That relieves the patients and increases confidence. Therefore, the system saves medical work time, optimizes the patient's medical experience, and also reduces medical disputes.

### BRIEF DESCRIPTION OF THE APPENDED DRAWINGS

FIG. 1 shows a schematic diagram in an embodiment of the system in the present invention;

FIG. 2 shows a schematic diagram in another embodiment of the system in the present invention;

FIG. 3 shows a schematic diagram in another embodiment of the system in the present invention;

FIG. 4 shows a schematic diagram in another embodiment of the system in the present invention;

FIG. 5 shows a schematic diagram in another embodiment of the system in the present invention;

FIG. 6 shows a schematic diagram in another embodiment of the system in the present invention;

FIG. 7 shows a schematic diagram of the report generation model in an embodiment of the system in the present invention;

FIG. 8 shows a schematic diagram of the customized medical information generated by the report generation model according to the embodiment of FIG. 7.

### DETAILED DESCRIPTION OF THE INVENTION

For the sake of the advantages, spirits and features of the present invention can be understood more easily and clearly, the detailed descriptions and discussions will be made later by way of the embodiments and with reference of the diagrams. It is worth noting that these embodiments are merely representative embodiments of the present invention, wherein the specific methods, devices, conditions, materials and the like are not limited to the embodiments of the present invention or corresponding embodiments. Moreover, the devices in the figures are only used to express their corresponding positions and are not drawing according to their actual proportion.

The terms used in the various embodiments disclosed in this invention are only used for the purpose of describing specific embodiments, and are not intended to limit the various embodiments disclosed in this creation. The singular form as used herein also includes the plural form, unless the context clearly indicates otherwise. The technical and scientific terms used in this specification have the same meanings as commonly understood by those skilled in the art if there is not further defined. The terms (such as those defined in commonly used dictionaries) will be interpreted as having the same meaning as the contextual meaning in the same technical field, and will not be interpreted as having idealized or overly formal meanings, Unless clearly defined in the various embodiments disclosed in the present creation. In the diagram, the system is represented by the label S.

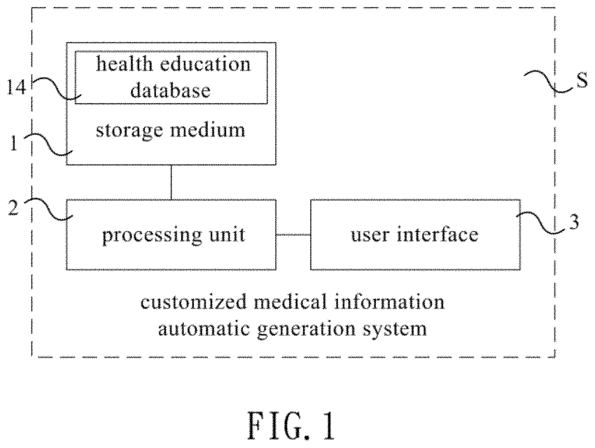

Please refer to FIG. 1. FIG. 1 shows a schematic diagram in an embodiment of the system in the present invention. The present invention provides a system for automatic generating customized medical information, comprising a storage medium 1, a processing unit 2, and a user interface 3. The storage medium 1 has a health education database 14. Wherein, the health education database 14 stores a plurality of health education information corresponding to a plurality of medical conditions. The processing unit 2 is coupled to the health education database 14. Wherein, according to a first medical condition described in diagnostic result information, the processing unit 2 selects a first health education information corresponding to the first medical condition from the health education database 14, and generates a customized medical information comprising the first medical condition and the first health education information. The user interface 3 is coupled to the processing unit 2 to receive and display the customized medical information.

Wherein, the storage medium 1 can be a cloud server, a local server, a floppy disk, an optical disc, a hard disk, a memory card, or a flash drive. Medical conditions include defined disease names such as gastric cancer, anemia, AIDS, myocardial infarction, fracture, appendicitis, kidney stones, urticaria, conjunctivitis, stroke, or diabetes. Health education information is applied to corresponding diseases, such as “cirrhosis” is correspond to “cirrhosis is caused by diffuse degeneration and necrosis of liver cells, fibrous tissue proliferation and nodular regeneration of liver cells, and the structure of liver lobules and blood circulation are gradually destroyed. It deforms and hardens the liver . . . The cause of liver cirrhosis is viral . . . The symptoms and complications are . . . The most common group is . . . The treatment is . . . ” The diagnostic result information may come from other diagnostic equipment, or the diagnostic made by the doctor during the consultation, and input into this system.

In the past, health education information would be printed on paper and placed in the open space of the hospital or clinic for patients to obtain by themselves, or provided by medical staff on their own initiative. However, there are numerous types of medical conditions, and it is difficult to print them all out on paper, except for several common or serious diseases. This leads to a lack of health education for rare diseases and makes patients feel uneasy. In this invention, the patients directly see the health education information most relevant to their own illness as long as the user interface is used, and there is no need to request a paper copy. In addition to being convenient and intuitive, it also saves energy and protect environment.

The system also allows medical units to provide personalized medical information and reports. The medical personnel fulfill their obligation to inform, and patients get the right to know, and there are clear guarantees for both them all. Providing customized medical information can make patients feel easier and their disease is being paid attention, rather than the same mechanized medical treatment order.

Wherein, the storage medium 1 stores a command, and the processing unit 2 is configured to execute the command to select the first health education information according to the diagnostic result information and generate the customized medical information.

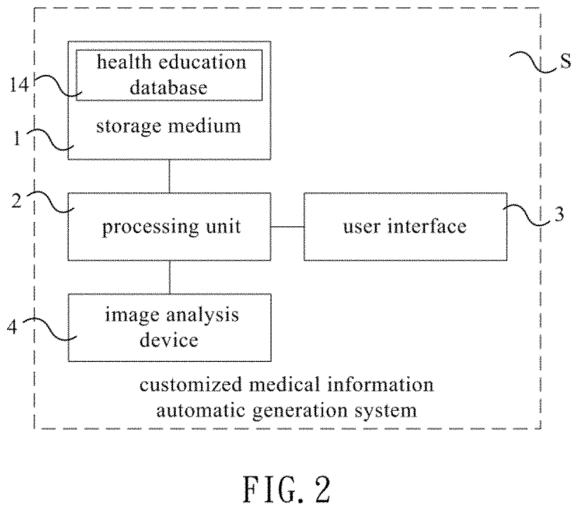

Please refer to FIG. 2. FIG. 2 shows a schematic diagram in another embodiment of the system in the present invention. In this embodiment, objects and actions not specifically mentioned are the same as the previous embodiment in principle. The system for automatic generating customized medical information further comprises an image analysis device 4. The image analysis device 4 is coupled to the processing unit 2. Wherein, the image analysis device 4 is configured to analyze a medical image corresponding to a patient to diagnosis the first medical condition, and to output the diagnostic result information comprising the first medical condition and the medical image. The processing unit 2 is configured to generate the customized medical information comprising the medical image, the first medical condition and the first health education information. Medical image such as optical photographs, photos ultrasound, X-ray, tomography, nuclear magnetic resonance, and positron tomography is obtained. The automatic instrument parameter setting, machine learning, or deep learning is used to make the image analysis device 4 be able to determine the symptoms represented in the medical image. The image analysis device 4 may use several medical images simultaneously to complete the diagnosis of one or several diseases of a patient. Then, the image analysis device 4 can output the name of the disease and the medical image for diagnosis to the processing unit as the diagnosis result information.

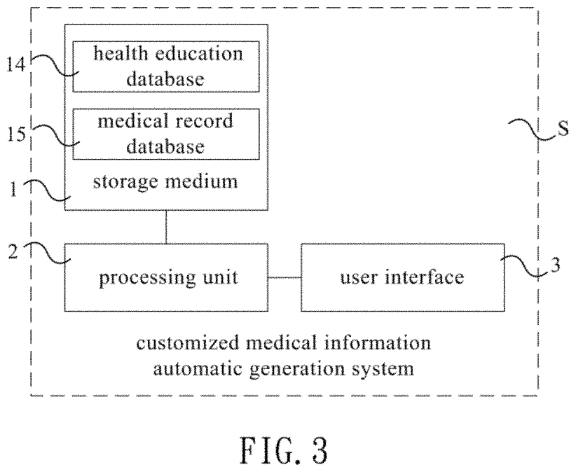

Please refer to FIG. 3. FIG. 3 shows a schematic diagram in another embodiment of the system in the present invention. In this embodiment, objects and actions not specifically mentioned are the same as the previous embodiment in principle. The storage medium 1 of the system further comprises a medical record database 15. The medical record database 15 is coupled to the processing unit 2, wherein the medical record database 15 stores basic information of a patient and at least one medical history information. Wherein, the processing unit 2 is configured to adjust the customized medical information according to the basic information and the medical history information. Each of the health education information comprises a prognosis information, a symptom information, a medical care information, and a medical order information. Each of the health education information is divided into a plurality of differential health education information according to a clinical difference factor. Most of the plurality of differential health education information of the same health education information has the same or similar prognosis information, symptom information, medical care information, and medical order information, a small part of the information has different description.

The processing unit 2 is configured to compare the clinical difference factors with the basic information of the patient and the medical history information, and to select a first differential health education information correspondingly from the first health education information to generate the customized medical information. The basic information of the patient includes gender, age, blood type, height, weight, etc. When the patient is not the first visit, or there is a commonality database for accessing, the past medical information of the patient will be referred, such as suffering from high blood pressure three years ago, performing cardiac catheterization a year ago, and having a history of allergies to antibiotics.

Clinical difference factors include age, gender, pregnancy, etc., so some health education information must be adjusted. For example, patients who are 20 weeks pregnant should not be recommended for swimming and running, but other recommendations should be given. The health education information is divided into multiple differential health education information based on clinical difference factors. For example, “diabetes” is divided into “type 1 diabetes health education information” and “type 2 diabetes health education information” according to “age”, “weight”, and “family medical history” . . . etc.

The clinical difference factors been focused on by each type of health education information are not necessarily the same. For example, “urethritis” will classify differential health education information because of “antibiotic allergy”, but “Parkinson's disease” will not be classified because of “antibiotic allergy”.

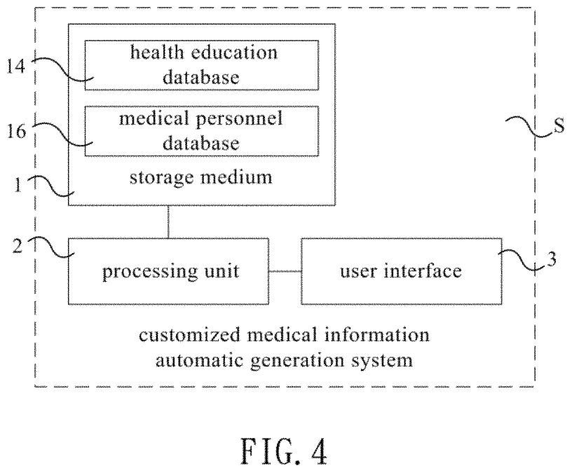

Please refer to FIG. 4. FIG. 4 shows a schematic diagram in another embodiment of the system in the present invention. In this embodiment, objects and actions not specifically mentioned are the same as the previous embodiment in principle. The storage medium 1 of the system further comprises a medical personnel database 16. The medical personnel database 16 is coupled to the processing unit 2 and storing a medical personnel information of a medical personnel. Wherein, the processing unit 2 is configured to select the medical personnel information of the medical personnel corresponding to a patient form the medical personnel database 16. The customized medical information generated by the processing unit 2 comprises the medical personnel information.

Medical personnel include the patient's attending physician, nurse practitioner, responsible resident, technical team, unit supervisor, etc. Medical personnel information can include academic experience, expertise, contact methods, etc. Medical personnel information may be presented in photos or videos of medical staff. Providing medical personnel information shows the responsibility of the medical unit, and patients will be relatively more relieved.

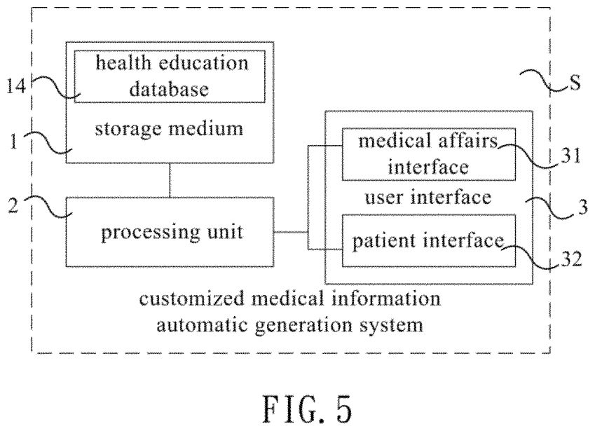

Please refer to FIG. 5. FIG. 5 shows a schematic diagram in another embodiment of the system in the present invention. In this embodiment, objects and actions not specifically mentioned are the same as the previous embodiment in principle. The user interface 3 of the system further comprises a medical affairs interface 31. The medical affairs interface 31 is coupled to the processing unit 2 for inputting a customized medical order; wherein, the processing unit 2 is configured to adjust the customized medical information according to the customized medical order.

Although the storage medium 1 may store numerous diseases and corresponding health education information, and the system may generate corresponding customized medical information automatically, medical personnel still have the ability and responsibility to review and judge. With the medical affairs interface 31, medical personnel can further edit and modify customized medical information and health education information templates, and even save them as new health education information templates.

On the other hand, the user interface 3 of the system further comprises a patient interface 32. The patient interface 32 is coupled to the processing unit 2 to receive and display the customized medical information. In principle, the customized medical information cannot be modified through the patient interface 32, and the patient is only allowed to use and be informed. However, the patient interface 32 allows the patient to give feedback or make an appointment for the next consultation in another input pane after customized medical information.

The patient interface 32 stops displaying customized medical information after a preset time, such as 2 weeks, 3 months, or 2 years. That is because the patient's condition may change due to time, so that the current customized medical information also has its applicable period. The customized medical information out of date may make patients have a wrong perception of the latest condition. In specific applications, the past customized medical information is stored in the medical record database 15 still without being deleted, which only prevents the patient from viewing it through the patient interface 32, but the medical staff can view it from the medical interface 31.

The patient interface 32 is a web browser interface or a mobile application interface. Patients may connect to the exclusive customized medical information page by URL link or QR code. The patient interface 32 may also be made into account login, allowing patients to enter their account password and jump to the exclusive customized medical information page. Or the patient interface 32 may be a mobile application interface APP, through the mobile phone account binding, the exclusive customized medical information will be displayed after opening the APP.

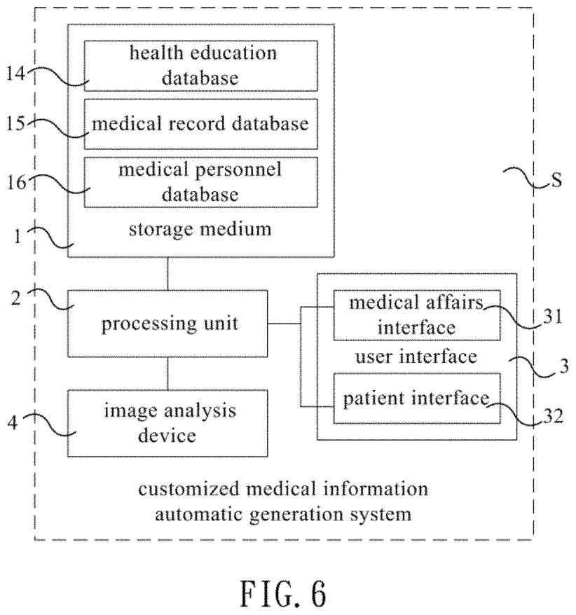

Please refer to FIG. 6. FIG. 6 shows a schematic diagram in another embodiment of the system in the present invention. In this embodiment, objects and actions not specifically mentioned are the same as the previous embodiment in principle. The aforementioned objects and actions can be implemented in full or in part if they do not conflict with each other.

When the image analysis device 4 receives at least one medical image, the image analysis device 4 sends the name of the disease and the image to the processing unit 2. The processing unit 2 finds out the health education information corresponding to the disease from the health education database 14. The processing unit 2 compares the basic data of the patient in the medical record database 15 and the past medical information with the clinical difference factors of the health education information, and selects the appropriate differential health education information. Then the processing unit 2 picks out the medical personnel information corresponding to the patient from the medical personnel database 16. Furthermore, all the symptoms, medical images, differential health education information, medical personnel information, and so forth are presented on the user interface 3. Especially, the medical personnel may review, adjust instructions, and add orders through the medical interface 31 to form optimized and accurate customized medical information. At last, the customized medical information is presented to the patient by the patient interface 32.

In addition, the system of this invention may further include a recording device (not shown) coupled to the medical record database 15. When medical staff discusses the condition of the patient in the clinic, the recording device of this system records and stores the voice in the medical record database 15. The patient interface 32 provides recorded messages with generating customized medical information, so that patients can recall the content of the discussion afterwards.

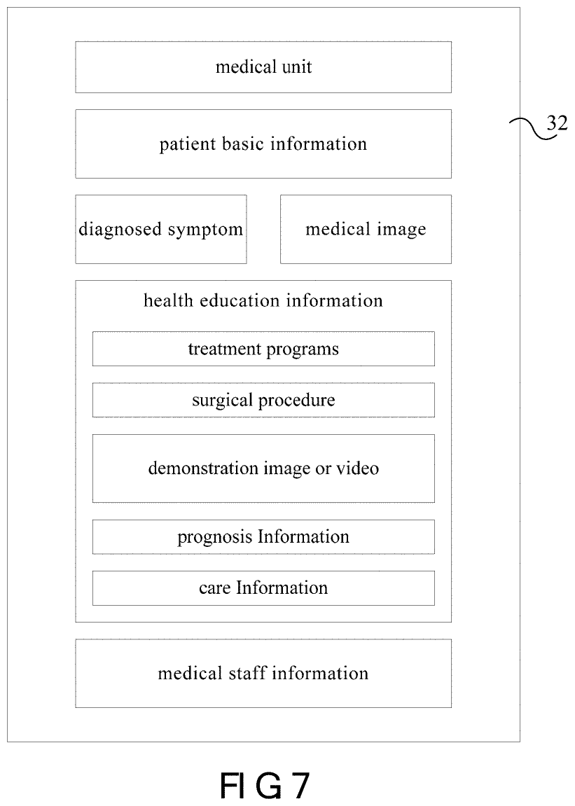

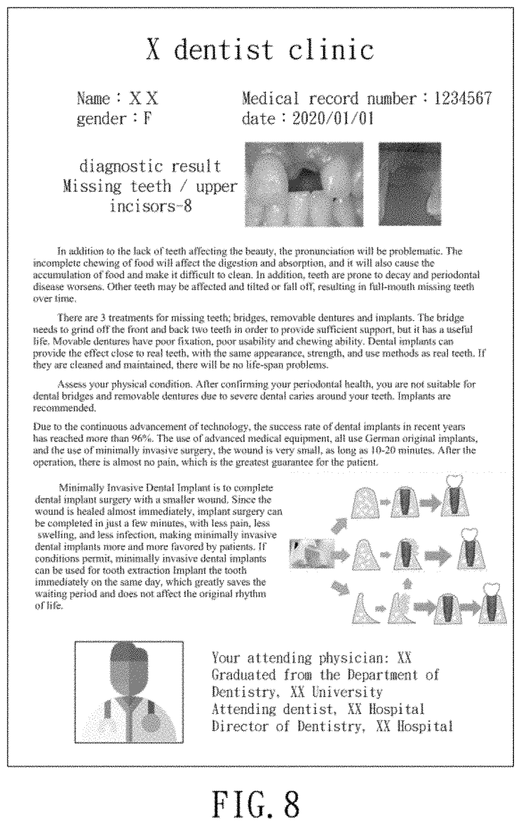

Please refer to FIG. 7 and FIG. 8. FIG. 7 shows a schematic diagram of the report generation model in an embodiment of the system in the present invention; FIG. 8 shows a schematic diagram of the customized medical information generated by the report generation model according to the embodiment of FIG. 7. In this embodiment, objects and actions not specifically mentioned are the same as the previous embodiment in principle.

The storage medium 1 of the system further comprises a report generation model. The processing unit 2 is configured to generate the customized medical information into a customized medical report according to the report generation model. The user interface 3, especially the patient interface 32, is configured to present the customized medical report. The report generation model is the basic framework of customized medical reports, and the processing unit 2 needs to find the corresponding data to fill in the framework. Customized medical report refers to the presentation of customized medical information in a formal or printable format.

In the embodiment of FIG. 8, a dental diagnosis report is taken as an example. At the top of the report, basic patient information, diagnosed symptoms, and medical images are provided. Health education information is provided in the middle of the report, including text, icons, or videos. Especially in the report, because of the clinical difference of the patients, “peripheral dental caries”, it is recommended to treat dental decay by means of dental implants, so there is no need to explain the precautions of dental bridges or movable dentures. At the bottom of the report, the doctor's medical staff information including academic experience is provided, or a video or voice recorded by the medical staff may be used as a supplement.

In summary, the system provided in the present invention greatly reduces the time required for medical staff to prepare medical reports, and especially customizes for patients with different conditions. From the patient's point of view, seeing complete, customized, and easy-to-understand health education information will help to understand their own disease, clearly choose treatment options and take care of body. In particular, patients also see demonstrations, predictions, and attending physicians or related medical personnel from the user interface. That relieves the patients and increases confidence. Therefore, the system saves medical work time, optimizes the patient's medical experience, and also reduces medical disputes.

With the examples and explanations mentioned above, the features and spirits of the invention are hopefully well described. More importantly, the present invention is not limited to the embodiment described herein. Those skilled in the art will readily observe that numerous modifications and alterations of the device may be made while retaining the teachings of the invention. Accordingly, the above disclosure should be construed as limited only by the metes and bounds of the appended claims.

What is claimed is:

1. A system for automatic generating customized medical information, comprising:
a storage medium, having a health education database, wherein the health education database stores a plurality of health education information corresponding to a plurality of medical conditions;
a processing unit, coupled to the health education database, wherein according to a first medical condition described in a diagnostic result information, the processing unit selects a first health education information corresponding to the first medical condition from the health education database, and generates a customized medical information comprising the first medical condition and the first health education information; and
a user interface, coupled to the processing unit to receive and display the customized medical information.

2. The system for automatic generating customized medical information of claim 1, further comprising:
an image analysis device, coupled to the processing unit, wherein the image analysis device is configured to analyze a medical image corresponding to a patient to diagnosis the first medical condition, and to output the diagnostic result information comprising the first medical condition and the medical image.

3. The system for automatic generating customized medical information of claim 2, wherein the processing unit is configured to generate the customized medical information comprising the medical image, the first medical condition and the first health education information.

4. The system for automatic generating customized medical information of claim 1, wherein the storage medium further comprises:
a medical record database, coupled to the processing unit, wherein the medical record database stores a basic information of a patient and at least one medical history information;
wherein, the processing unit is configured to revise the customized medical information according to the basic information and the medical history information.

5. The system for automatic generating customized medical information of claim 4, wherein each of the health education information comprises a prognosis information, a symptom information, a medical care information, and a medical order information, each of the health education information is divided into a plurality of differential health education information according to a clinical difference factor, the processing unit is configured to compare the clinical difference factors with the basic information of the patient and the medical history information, and to select a first differential health education information correspondingly from the first health education information to generate the customized medical information.

6. The system for automatic generating customized medical information of claim 1, wherein the storage medium further comprises:
a medical personnel database, coupled to the processing unit and storing a medical personnel information of a medical personnel;
wherein, the processing unit is configured to select the medical personnel information of the medical personnel corresponding to a patient form the medical personnel database, and the customized medical information generated by the processing unit comprises the medical personnel information.

7. The system for automatic generating customized medical information of claim 1, wherein the user interface further comprises:
a medical affairs interface, coupled to the processing unit for inputting a customized medical order;
wherein, the processing unit is configured to revise the customized medical information according to the customized medical order.

8. The system for automatic generating customized medical information of claim 1, wherein the user interface further comprises:
a patient interface, coupled to the processing unit to receive and display the customized medical information, wherein the patient interface stops displaying the customized medical information after a preset period, and the patient interface is a web browser interface or a mobile application interface.

9. The system for automatic generating customized medical information of claim 1, wherein the storage medium further comprises:
a report generation model, wherein the processing unit is configured to generate the customized medical information into a customized medical report according to the report generation model, and the user interface is configured to present the customized medical report.

10. The system for automatic generating customized medical information of claim 1, wherein the storage medium stores a command, and the processing unit is configured to execute the command to select the first health education information according to the diagnostic result information and generate the customized medical information.
